(19) United States
(12) Patent Application Publication
(10) Pub. No.: US 2014/0093451 A1
(43) Pub. Date: Apr. 3, 2014

(54) Coupled identification and treatment of cancer

(71) Applicant: Lawrence Richard Bernstein, Menlo Park, CA (US)

(72) Inventor: Lawrence Richard Bernstein, Menlo Park, CA (US)

(21) Appl. No.: 14/095,066

(22) Filed: Dec. 3, 2013

Related U.S. Application Data
(63) Continuation of application No. 13/256,118, filed on Sep. 12, 2011.
(60) Provisional application No. 61/167,282, filed on Apr. 7, 2009.

(51) Int. Cl. A61K 51/04; A61K 51/02

(52) U.S. Cl. CPC A61K 51/0472; A61K 51/025
USPC 424/1.61; 424/1.65

(57) ABSTRACT

Provided are methods to treat cancer, in which (1) a patient is first identified as having a cancer that is likely to be susceptible to gallium therapy, by the use of a gallium scan or other procedure that shows whether the cancer is gallium-avid, and (2) the patient is then treated with a pharmaceutically acceptable gallium composition.

### CROSS REFERENCE TO RELATED PATENT APPLICATIONS

This application is a continuation of U.S. application Ser. No. 13/256,118, filed Sep. 12, 2011, which is the U.S. National Phase under 35 U.S.C. §371 of International Application No. PCT/US2010/030054, filed Apr. 6, 2010, which claims priority under 35 U.S.C. §119(e) to U.S. Provisional Application No. 61/167,282, filed Apr. 7, 2009, the disclosure of each application being incorporated by reference herein in its entirety.

### TECHNICAL FIELD

This invention pertains generally to treatments for cancer. More particularly, this invention pertains to identifying a patient who has gallium-avid cancer by using a gallium scan or other means, and then treating the patient with a pharmaceutically acceptable gallium composition.

### BACKGROUND OF THE INVENTION

Gallium radioisotopes, particularly 67Ga, have been in widespread use since about 1969 to help detect and localize cancer, infection, and inflammation in the body. The detection and localization are typically accomplished with a gallium scan. In this method, a small amount of ⁶⁷Ga citrate is administered intravenously, and then one or more scans are performed using a suitable radiation detector to map the distribution of ⁶⁷Ga in the body. All or some of the scans are commonly performed following a waiting period, generally of about 18 to 96 hours, to allow time for ⁶⁷Ga uptake and for clearance of some ⁶⁷Ga from the gastrointestinal tract, blood, and healthy tissues. Scans may be made of the entire body or of selected portions of the body. The scans may produce planar (2-D) data or three-dimensional (3-D) data, the latter generally derived from single-photon emission computerized tomography (commonly abbreviated as SPECT or SPET); planar and 3-D data are commonly gathered in a single session. If gallium-avid cancer tissue is present, it will become more radioactive than healthy surrounding tissue, and the contrast in radioactivity between the pathological tissue and surrounding healthy tissue will be detectable in the planar or SPECT scan. Decades of gallium scan results show that little gallium is taken up by most healthy tissues, even by those containing rapidly multiplying cells (such as the stomach lining, bone marrow, and hair follicles). Small to moderate uptake is, however, sometimes observed in normal tissues, particularly liver, growth plates of bones in children and adolescents, intestines (where some gallium may be excreted), nasopharyngeal region, lacrimal glands, salivary glands, breast (especially lactating), thymus, and spleen.

Gallium, in its naturally occurring, non-radioactive form, is known to be effective in treating many types of cancer. In vitro, animal, and human studies have shown, for example, that gallium can be effective against lymphoma, multiple myeloma, prostate cancer, bladder cancer, liver cancer, breast cancer, cervical cancer, medulloblastoma, lung cancer, ovarian cancer, colon cancer, and other cancers. One mechanism of action for gallium appears to be its ability to act as an irreducible mimic of ferric iron (Fe³⁺), and as such to interfere with the uptake and utilization of iron by pathologically proliferating cells. Pathologically proliferating cells, including cancer cells, must acquire ferric iron in order to multiply; this is because ferric iron is needed in the active site of ribonucleotide reductase, an enzyme essential to the synthesis of DNA. Therefore, in many cases, Ga³⁺ is avidly taken up by cancer cells (as well as by many bacteria, other pathogens, and other pathologically proliferating cells). The gallium thus taken up may then interfere with the utilization of iron within the cell, inhibiting DNA synthesis and cell division.

It has now been discovered that gallium scanning can identify those patients who have cancers that are most likely to be susceptible to gallium therapy (gallium-responsive cancers). Gallium-avid cancer, as identified by a gallium scan or other means, is likely to take up therapeutically administered gallium; the gallium will then inhibit the growth of the cancer, leading to stabilization, reduction, or elimination of the cancer. Thus, a cancer that is gallium avid is also very likely to be gallium-responsive. The ability to screen for, image, and then treat a disorder all with the same chemical entity—in this case gallium—constitutes a powerful new method of identifying and treating disease. Although this invention is focused on the treatment of cancer, the same principles of identification and treatment can be applied to infections, inflammations, and other pathological conditions that are avid for and treatable by gallium. Similarly, the same principles can be applied to agents other than gallium, when they are used for both diagnosis and treatment.

### SUMMARY OF THE INVENTION

Accordingly, it is a primary object of the invention to provide methods for treating cancer.

In an embodiment of the invention, a method is provided for treating cancer comprising identifying a patient whose cancer can take up gallium and administering to the patient thus identified a therapeutically effective amount of a pharmaceutically acceptable gallium compound.

In another embodiment, a method is provided for treating cancer comprising identifying a patient who has cancer detectable by a gallium scan and administering to the patient thus identified a therapeutically effective amount of a pharmaceutically acceptable gallium compound.

In another embodiment, a method is provided for treating cancer comprising identifying a patient who has cancer detectable by a gallium scan and administering to the patient thus identified a therapeutically effective amount of gallium maltolate.

In another embodiment, a method is provided for identifying a cancer patient whose cancer is responsive to treatment with gallium comprising: a) determining if the cancer tissue can take up gallium, and then b) identifying the cancer patient as being responsive to treatment with gallium when the cancer tissue is determined to take up gallium.

In another embodiment, a method is provided for identifying a tumor as responsive to treatment with gallium comprising: a) determining if the tumor can take up gallium, and then b) identifying the tumor as responsive to treatment with gallium when the tumor is determined to take up gallium.

In another embodiment, a composition is provided comprising a pharmaceutically acceptable gallium compound for the treatment of a gallium-responsive cancer, wherein the cancer is identified as being gallium-responsive by a method comprising: a) determining if the cancer can take up gallium, and then b) identifying the cancer as responsive to treatment with gallium when the cancer is determined to take up gallium.

### DETAILED DESCRIPTION OF THE INVENTION

Before the present methods of the invention are disclosed and described, it is to be understood that this invention is not limited to specific formulations (e.g., specific carrier materials or the like), to specific dosage regimens, or to specific drug delivery systems, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a gallium compound” includes mixtures of such compounds; reference to “a carrier” includes mixtures of two or more carriers; and the like.

The terms “patient” and “subject” are meant to include a human or a veterinary patient or subject. Within the context of the present invention, veterinary patients are intended to include both mammalian and non-mammalian veterinary patients, the latter including such veterinary patients as, for example, lizards and birds.

The terms “active agent,” “drug,” and “pharmacologically active agent” are used interchangeably herein to refer to a chemical material or compound that, when administered to a patient, induces a desired pharmacologic effect, such as treatment of cancer.

The term “effective” in reference to the amount of a drug means that there is a sufficient amount of a compound to provide the desired effect and performance at a reasonable benefit/risk ratio attending any medical treatment.

The term “gallium-responsive”, as in “gallium-responsive cancer”, means that gallium is effective for treatment, as of the cancer.

This invention pertains to a method for treating cancer comprising identifying a patient whose cancer can take up gallium and administering to the patient thus identified a therapeutically effective amount of a pharmaceutically acceptable gallium compound. The therapeutically effective amount is an amount effective to inhibit growth of the cancer of the patient and/or reduce symptoms of the cancer, such as pain.

Treatment is applicable to human and veterinary patients, including particularly mammals and birds. Mammalian veterinary subjects include, without limitation, dogs, cats, and members of the families Equidae, Bovidae, Caprinae, and Suidae. Veterinary subjects also include, without limitation, reptiles, amphibians, and fish.

In a preferred embodiment, identifying a patient is accomplished by use of a gallium scan on the patient. The methods of performing gallium scans on patients are well known in the art (see, for example, Goldsmith S J et al., Gallium-67 imaging for the detection of malignant disease, in Sandler M P et al., eds., Diagnostic Nuclear Medicine, Fourth Edition. Philadelphia: Lippincott Williams & Wilkins, 2003, pp. 913-929; and Bartold S P et al., Procedure Guideline for Gallium Scintigraphy in the Evaluation of Malignant Disease, Journal of Nuclear Medicine 38:990-994, 1997). Thousands of published references regarding gallium scans can be found in the literature.

Very briefly, a gallium scan is performed by administering a small amount of a gallium radioisotope (usually ⁶⁷Ga) to a subject and then scanning the subject to map the distribution of resulting radioactivity in the body; the amount of radioactivity will be directly proportional to the uptake of gallium. Scanning is performed on the whole body or portions of the body using a scintillation detector or other suitable radiation detector.

The most commonly used gallium radioisotope, ⁶⁷Ga, has a half life of about 78.3 hours. It is most readily available as ⁶⁷Ga citrate, though other compounds may be prepared and used. ⁶⁷Ga decays by electron capture to stable ⁶⁷Zn, emitting predominately gamma rays at principal energy values of about 93.3, 184.6, 300.2, and 393.5 KeV. If ⁶⁷Ga is used, then the radiation detector used for scanning must be able to detect one or more of these energies of gamma rays. The amount of ⁶⁷Ga administered to an adult weighing about 70 Kg is generally about 74-370 MBq (2-10 mCi) (or about 1-5 MBq per Kg of body weight), though other dose levels may be administered. Administration is generally by intravenous injection.

Scans may be made at any time following administration of the gallium radioisotope, though it is commonly advantageous to wait from several hours to about 96 hours, or more, before performing one or more of the scans. This waiting time allows some of the gallium that is not taken up by body tissues, particularly by the pathological tissues or cells of interest, to be excreted from the body; higher contrast between regions of gallium uptake and other regions of the body is thus permitted. The waiting period is particularly helpful for imaging the abdominal area, because some gallium is generally excreted by the intestines, and normal liver may transiently take up some gallium. If abdominal areas are imaged, contents of the gastrointestinal tract, or at least the large intestine, are sometimes intentionally cleared; this is accomplished by administering a laxative and/or enema shortly before performing a scan. This bowel clearance reduces the amount of radioactive gallium that may have accumulated in this region, which otherwise could interfere with observations of abdominal organs and tissues.

Scanning is performed using a scintillation detector or another detector that is sensitive to the radiation produced by the gallium radioisotope (e.g., gamma rays for ⁶⁷Ga). For ⁶⁷Ga, a multipeak gamma camera with a large field of view and head shielding is commonly used. Scans may be either planar (two-dimensional (2-D) imaging) or as multiple tomographic scans leading to three-dimensional (3-D) imaging. The latter scans generally employ single-photon emission computerized tomography (SPECT or SPET), which may provide higher contrast and localization than planar images alone.

The uptake of ⁶⁷Ga (or other gallium radioisotopes) by cancer tissue may be quantified or semi-quantified using methods known in the art (see, for example, Lin W Y et al., Eur J Nucl Med 27(11): 1626-1631, 2000; and Chang C S et al., Rheumatol Int 23(4): 178-181, 2003). Very briefly, the method of Lin et al. (2000) is as follows: This semi-quantitative method compares ⁶⁷Ga concentrations in tumors to those in nearby, healthy tissue of the same type, or of other healthy nearby tissue. Regions of interest (ROI) are drawn (or otherwise identified) around tumors and around regions of healthy tissue. The mean counts-per-pixel (or counts per unit area) are measured for each ROI, and the ratios of the tumor values to the non-tumor values are recorded. Analyses can be made for a sum of all target tumors and/or for the largest tumor alone. Very briefly, the method of Chang et al. (2003) is as follows: The radiation intensity recorded for a tumor is quantitatively compared to that for a standard. The weight of ⁶⁷Ga solution injected into the subject is recorded. A ⁶⁷Ga standard is prepared from an aliquot of the injection solution diluted 50-fold. A 1 mL portion of this solution is placed next to the subject, at the approximate height of the tumor, in a position that does not affect the gallium scan. In the ⁶⁷Ga scan, regions of interest are drawn (or otherwise identified) around the tumor(s) and around the standard, and counts and pixel numbers are recorded for each. ⁶⁷Ga relative concentration is calculated as follows: (tumor count/standard count)×(weight of standard×10,000/weight of injection×50 [dilution factor]). Analyses can be made for a sum of all target tumors and/or for the largest tumor alone.

In one embodiment of the invention, the uptake of ⁶⁷Ga (or other gallium radioisotope) by cancer tissue is at least approximately 10% higher than that of nearby healthy tissue. In another embodiment, the uptake of ⁶⁷Ga (or other gallium radioisotope) by cancer tissue is at least approximately twice as high as that of nearby healthy tissue. In a further embodiment, the uptake of ⁶⁷Ga (or other gallium radioisotope) by cancer tissue is at least approximately ten times as high as that of nearby healthy tissue. In another embodiment, the uptake of ⁶⁷Ga (or other gallium radioisotopes) by cancer tissue is at least approximately one hundred times as high as that of nearby healthy tissue. In yet another embodiment of this invention, any visually discernable excess of ⁶⁷Ga (or other gallium radioisotope) uptake by cancer tissue relative to surrounding healthy tissue as observed in a gallium scan is sufficient.

In a preferred embodiment of this invention, approximately 74-370 MBq (2-10 mCi) of ⁶⁷Ga citrate is administered intravenously to a 70 Kg adult. For human or veterinary subjects of other weights, the amount of ⁶⁷Ga citrate administered is approximately 1-5 MBq per Kg of body weight. Then, scans are conducted at about 4 to about 240 hours after the ⁶⁷Ga citrate is administered, preferably at about 24-72 hours. For abdominal imaging, the contents of the lower gastrointestinal tract may be voided by fasting, laxative use, enema, or any combination of these methods, before a scan is performed; a preferred method is to administer 10 to 20 mg of bisacodyl the evening before a scan, followed by a Fleet enema the next morning, within a few hours of a scan. In the gallium scans, regions of interest, corresponding to one or more locations of tumors or other sites of cancerous tissue, are selected (by their observed radioactivity due to the gallium radioisotope, and/or from x-ray images, computed tomography (CT) images, magnetic resonance images (MRI), positron emission tomography (PET) images, or other imaging or cancer-localizing methods that cover the same region). The radioactivity due to the gallium radioisotope, as measured by visual inspection of scan image(s), count rates, optical densitometry on scan images, or similar means (such as those presented by the Lin et al., 2000 and Chang et al., 2003 references previously cited, or other literature on the quantitative or semi-quantitative analysis of gallium scans) is then recorded for the regions of interest (this corresponds to the uptake of the gallium radioisotope by the cancerous tissue) and compared with that for nearby healthy tissues. The comparison between cancerous tissue and healthy tissue on gallium scans may be done by visual inspection or by using quantitative or semiquantitative methods such as those just mentioned.

It is noted that this invention is not restricted to particular gallium radioisotopes, compounds, means of administration, or detection methods; under suitable circumstances, the gallium radioisotope may be incorporated in a variety of compounds and may be administered by a variety of routes, including oral, subcutaneous injection, intramuscular injection, peritoneal injection, and so on, and the radiation may be detected by any suitable radiation-detecting means.

Any alternate means of assaying the uptake of gallium by the pathological tissue (or other tissue or cells of interest) may also be employed in the practice of the invention. One such method involves removing cells or tissue of interest from the subject and bringing these into contact with a gallium-containing composition in solution. Preferred gallium-containing compositions for such a solution are gallium nitrate, gallium chloride, gallium sulfate, gallium citrate, and gallium transferrin. After contacting the cells with the gallium-containing solution for a period of about five minutes to about six hours, preferably about two hours, the cells are isolated by filtration and/or centrifugation, washed with water or other suitable washing material, and assayed for gallium content. Any suitable gallium assay method may be used; a preferred assay method is to dissolve the cells or tissue using nitric acid or other suitable solvent and then analyze the resulting solution using inductively coupled plasma mass spectrometry (ICP-MS). If the gallium content of the cells or tissue is higher than that of the solution in which they were exposed to gallium, then preferential uptake has occurred. Such preferential uptake is an indication to administer gallium to the subject for therapeutic purposes. In a closely related method, the cells or tissue of interest is exposed to a gallium composition in solution comprising a gallium radioisotope, preferably ⁶⁷Ga; again, preferred gallium compositions are gallium nitrate, gallium chloride, gallium sulfate, gallium citrate, and gallium transferrin. In this case, the assay is performed by isolating the cells by filtration and/or centrifugation, washing with water or other suitable washing material, drying the cells, and determining their radioactivity. If the radioactivity (per weight) is higher than that of the solution they were exposed to, then preferential uptake will have occurred.

Any pharmaceutically acceptable gallium compound may be used therapeutically in this invention, by any medically acceptable route of administration. Gallium compounds usable in this invention include, without limitation, gallium nitrate, gallium sulfate, gallium citrate, gallium chloride, gallium complexes of 3-hydroxy-4-pyrones including gallium maltolate, gallium tartrate, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexes of kenpaullone and its derivatives, and any other pharmaceutically acceptable gallium salts, organic salts, inorganic compounds, chelates, complexes, coordination compounds, and organometallic compounds. Gallium maltolate, tris(3-hydroxy-2-methyl-4H-pyran-4-onato)gallium, is a preferred gallium compound of the invention; this compound is described, for example, in U.S. Pat. No. 5,981,518 to Bernstein.

In one embodiment, the gallium compound is administered intravenously; for this purpose, gallium nitrate, gallium citrate, gallium palmitate, gallium porphyrins including gallium(III) protoporphyrin IX, gallium transferrin, bis(2-acetylpyridine 4N-dimethylthiosemicarbazone)gallium (III)-gallium(III) tetrachloride, pyridoxal isonicotinoyl hydrazone gallium(III), gallium maltolate, and gallium complexes of kenpaullone and its derivatives, in a suitable pharmaceutically acceptable liquid formulation, are preferred, with citrate-buffered gallium nitrate particularly preferred.

In other embodiments, the gallium compound may be injected directly into one or more tumors and/or blood vessels that directly feed the one or more tumors. The gallium compound may be injected into one or more tumors via intratumoral administration, which includes without limitation intratumoral injection and/or instillation. Injection of the gallium compound into one or more blood vessels, such as the hepatic artery or branches thereof, is useful for procedures such as for example, chemoembolization therapy. Gallium compounds useful for intratumoral administration and/or chemoembolization therapy include without limitation any of the following gallium compounds: gallium nitrate, gallium citrate, gallium palmitate, gallium porphyrins including gallium(III) protoporphyrin IX, gallium transferrin, bis(2-acetylpyridine 4N-dimethylthiosemicarbazone)gallium (III)-gallium(III) tetrachloride, pyridoxal isonicotinoyl hydrazone gallium(III), gallium maltolate, and gallium complexes of kenpaullone and its derivatives. Each of the gallium compounds set forth above is typically prepared in a suitable pharmaceutically acceptable formulation, such as a liquid or gel formulation. Gallium maltolate is a preferred gallium compound for use in intratumoral administration and chemoembolization therapy.

In a further embodiment, the gallium compound is administered orally. For this route of administration, preferred compounds are gallium nitrate, gallium citrate, gallium chloride, gallium 8-quinolinolate, and gallium maltolate; gallium maltolate is particularly preferred.

In other embodiments, the pharmaceutically acceptable gallium compound is administered topically, transdermally, per rectum, vaginally, buccally, subcutaneously, intramuscularly, peritoneally, into the ear, topical ocularly, intraocularly, by instillation into the bladder, urethrally, sublingually, using depot formulations and/or devices, or by any other safe and effective route known in the art of drug delivery. For topical, transdermal, rectal, vaginal, buccal, otic, topical ocular, intraocular, bladder, urethral, or sublingual delivery, gallium maltolate and gallium 8-quinolinolate are preferred compounds, with gallium maltolate being particularly preferred. For subcutaneous, intramuscular, or peritoneal delivery, gallium nitrate, gallium citrate, gallium maltolate, and gallium 8-quinolinolate are preferred compounds, with citrate-buffered gallium nitrate being particularly preferred.

The gallium compositions of the invention may also be formulated using liposomes. Such formulations may be particularly advantageous for sustained release or delayed release compositions.

The gallium compound is administered in a therapeutically effective amount, i.e., in an amount effective to inhibit growth of the cancer of the patient and/or reduce symptoms of the cancer, such as pain. Such amounts, when administered systemically, result in plasma gallium concentrations of about 1 to 10,000 ng/mL, preferably about 100 to 5,000 ng/mL, and most preferably about 500 to 2,000 ng/mL. Some non-limiting examples of therapeutically effective amounts are provided in the following four paragraphs.

When administered directly into a tumor or when used in chemoembolization therapy, the gallium concentrations of the injected liquid or gel are about 0.1 to about 10,000 μg/mL, preferably about 1.5 to 1,500 μg/mL, and more preferably about 100 to 1,000 μg/mL.

As an example of oral administration, gallium maltolate may be administered orally at a dose of about 50 to 5,000 mg/day, preferably about 200 to 3,000 mg/day, and more preferably about 300 to 2,000 mg/day, together with a pharmaceutically acceptable carrier. The dose may be administered in a single dose once per day, or in divided doses two or more times per day.

As an example of parenteral administration, citrate-buffered gallium nitrate is administered intravenously in a pharmaceutically acceptable intravenous liquid formulation, preferably as a slow infusion. The gallium nitrate is administered, for example, at a Ga(NO₃)₃ dose of about 10 to 1,000 mg/m²/day, preferably about 100 to 500 mg/m²/day, as a continuous intravenous infusion for about 1 to 10 days, preferably about 3 to 7 days. This dose may be repeated about every 1 to 12 weeks, preferably about every 2 to 4 weeks.

In an embodiment of the invention wherein the gallium compound is administered topically or otherwise locally, the gallium compound is present in a pharmaceutical formulation such that the gallium content is generally about 0.00001 percent to about 15 percent by weight of the formulation, preferably about 0.005 to about 1 percent, and most preferably about 0.02 to about 0.2 percent.

In one embodiment of the invention, a parenteral formulation of a gallium compound of the present invention is used in an improved intratumoral administration method by delivering the gallium compound directly into a tumor or lesion. In a preferred embodiment, the tumor or lesion is a hepatic tumor or lesion. In this method, the gallium compound, preferably gallium maltolate in a pharmaceutically acceptable liquid or gel carrier, is injected or otherwise instilled into the tumor or other lesion non-surgically or during surgery. The gel may contain pharmaceutically acceptable gel-forming materials such as, for example, soluble methylcellulose or carboxymethylcellulose, or purified bovine collagen. The gel delivery systems described, for example, in U.S. Pat. No. 6,630,168 to Jones et al.; U.S. Pat. No. 6,077,545 to Roskos et al.; U.S. Pat. No. 5,051,257 to Pietronigro; and U.S. Pat. No. RE 33,375 to Luck et al. may be used with the present invention. Additives, such as, for example, epinephrine as a vasoconstrictor to help retain the liquid or gel formulation within the tumor, may also be used.

In another embodiment of the invention, a parenteral formulation of a gallium compound, such as for example, gallium maltolate, is used in an improved chemoembolization method that uses the gallium compound to treat primary or metastatic liver cancer. In this method, the gallium compound, in a suitable pharmaceutically acceptable liquid or gel carrier, is injected into the hepatic artery or a branch of the hepatic artery feeding the region of the liver to be treated, together with standard embolization substances (such as certain oils and particulate matter; see, for example, Khayata et al., NEUROSURG CLIN N AM 5(3):475-484, 1994), which block arterial blood supply to the treated region. The rationale for this treatment is that normal liver tissue receives 75% of its blood supply from the portal vein and 25% from the hepatic artery, whereas liver tumors receive about 90% of their blood supply from the hepatic artery. Chemoembolization delivers a high dose of an antineoplastic drug directly to tumors, while simultaneously cutting off their subsequent arterial blood supply. Healthy liver tissue receives little exposure to the antineoplastic drug (such as gallium), and continues to receive the bulk of its normal blood supply, which comes from the portal vein. Chemoembolization formulations may include pharmaceutically acceptable oils, such as, for example, poppy seed oil or iodated poppy seed oil (e.g., lipiodol, to enhance radio-opacity). Biocompatible particulate matter may also be employed during chemoembolization; such particulate matter may comprise, for example, polyvinyl alcohol (PVA) (approximately 150-250 μm diameter) or tris-acryl gelatin microspheres (approximately 100-300 μm diameter). Typically, the gallium compound, such as gallium maltolate, will be administered in a water/oil emulsion; then, the particulate matter will be administered, commonly together with oil and/or radio-opaque material.

In another embodiment of the invention, the identified patient is administered a cytotoxic factor in addition to a pharmaceutically acceptable gallium compound. The cytotoxic factor may be any chemotherapeutic drug; a few such chemotherapeutic drugs are, as examples and without limitation, 5-fluorouracil, vinblastine, actinomycin D, etoposide, cisplatin, paclitaxel, methotrexate, and doxorubicin.

In a further embodiment of the invention, the identified patient is administered a monoclonal antibody directed at treating the cancer (such as, for example, anti-HER-2 antibodies or anti-CD20 antibodies), in addition to a pharmaceutically acceptable gallium compound.

In another embodiment of the invention, the identified patient is administered an anti-inflammatory drug in addition to a pharmaceutically acceptable gallium compound. The anti-inflammatory drug may be, without limitation, an anti-inflammatory steroid drug (such as, for example, dexamethasone or prednisone) or a non-steroidal anti-inflammatory drug (such as, for example, aspirin or ibuprofen; or COX-2 inhibitors, such as celecoxib).

In another embodiment of the invention, the identified patient is administered, in addition to the pharmaceutically acceptable gallium compound, one or more other anti-cancer agents, including, without limitation, growth inhibitory agents, agents used in radiation therapy, anti-angiogenesis agents, apoptotic agents, anti-tubulin agents, epidermal growth factor receptor (EGFR) antagonists (e.g., a tyrosine kinase inhibitor), HER1/EGFR inhibitors (e.g., erlotinib), platelet derived growth factor inhibitors (e.g., imatinib), interferons, cytokines, antagonists (e.g., neutralizing antibodies) that bind to, for example, one or more of the following targets ErbB2, ErbB3, ErbB4, PDGFR-beta, BlyS, APRIL, BCMA, or VEGF receptor(s), TRAIL/Apo2, antimetabolites (e.g., methotrexate), and so on.

The invention is not limited to the treatment of any particular type of cancer. Treatment of any cancer that takes up gallium is included in this invention. A few, non-limiting, examples of treatable cancers are primary liver cancers, breast cancers, lymphomas, bladder cancers, lung cancers, prostate cancers, myelomas, brain cancers, pancreatic cancers, colorectal cancers, osteosarcomas, cancers metastatic to the bone, melanomas, head and neck cancers, ovarian cancers, cervical cancers, gastric cancers, adenocarcinomas, sarcomas, and metastatic cancers. Pain associated with any cancer, particularly cancers that affect bone, is also treatable with this invention.

The practice of the present invention will employ, unless otherwise indicated, conventional techniques of drug formulation, which are within the skill of the art. Such techniques are fully explained in the literature. See, for example, REMINGTON: THE SCIENCE AND PRACTICE OF PHARMACY (Univ. of the Sciences in Philadelphia, 2000) as well as Goodman & Gilman's THE PHARMACOLOGICAL BASIS OF THERAPEUTICS, 9th Ed. (New York: McGraw-Hill, 1996) and Ansel et al., PHARMACEUTICAL DOSAGE FORMS AND DRUG DELIVERY SYSTEMS, 6^{th} Ed. (Media, Pa.: Williams & Wilkins, 1995).

All patents, patent documents, and non-patent publications cited herein are hereby incorporated by reference in their entirety for their disclosure concerning any pertinent information not explicitly included herein.

It is to be understood that while the invention has been described in conjunction with the preferred specific embodiments thereof, the foregoing description, as well as the example that follows, are intended to illustrate and not limit the scope of the invention. Other aspects, advantages and modifications will be apparent to those skilled in the art to which the invention pertains.

### EXPERIMENTAL

The following example is put forth so as to provide those of ordinary skill in the art with a complete disclosure and description of a non-limiting example of how to practice the invention. While efforts have been made to ensure accuracy with respect to variables such as amounts, temperature, etc., experimental error and deviations should be taken into account.

### Example 1

### Identification and Treatment of a Subject With Primary Liver Cancer

The subject of this study was a 69-year-old woman who was diagnosed with non-resectable primary liver cancer (hepatocellular carcinoma). The diagnosis was based on results of x-ray CT scans and tumor biopsy. Within two weeks of diagnosis the subject began treatment with Nexavar® (sorafenib) at a dose of 800 mg/day. The Nexavar® treatment was terminated after about 10 weeks due to the patient experiencing severe peripheral neuropathy, nausea, fatigue, gastrointestinal disorders, and anorexia.

Three weeks after Nexavar® treatment was terminated the subject had a gallium scan using 134 MBq of intravenously administered ⁶⁷Ga citrate. Planar and SPECT images were obtained 48 hours after ⁶⁷Ga citrate administration. These images showed intense gallium uptake in the liver tumors (average counts per second of approximately twenty to fifty times those in surrounding healthy liver tissue), with very low uptake in the surrounding liver tissue and in other organs. At that time the subject was experiencing moderate nausea, anorexia, and fatigue, with severe pain and tenderness of the right abdomen that prevented the subject from lying on her right side.

Based on the high avidity of the subject's hepatocellular carcinoma for gallium, as shown by the gallium scans, treatment of the patient with orally administered gallium maltolate was initiated. Treatment was started about a week after the gallium scans were performed. Gallium maltolate was administered as two 750 mg tablets taken once per day before breakfast (for a dose of 1500 mg/day). The largest tumor was about 20 cm in diameter by CT scan at three weeks before gallium maltolate administration was started.

Two weeks after the start of gallium maltolate treatment, measures of liver condition showed significant improvement; for example, serum bilirubin (total) dropped from 27.5 to 11. 9 μmol/L (normal: 2-20 μmol/L) and serum AST dropped from 132 to 70 IU/L (normal: 0-40 IU/L). The patient reported that her right abdominal pain was nearly gone, and she could lie and sleep on her right side. Her ability to engage in normal activities had substantially increased, so that she could now travel and go to concerts. Her condition continued to improve over the next six months. At about four months into the treatment, a CT scan showed no new tumor growth, with apparent necrosis of the primary tumor.

I claim:

1. A method of treating cancer in a patient in need thereof, comprising:
(1) administering to the patient a gallium radioisotope;
(2) performing a gallium scan on the patient;
(3) measuring the ratio of gallium radioisotope uptake in cancer tissue to that in nearby healthy tissue from the gallium scan;
(4) treating the patient with a therapeutically effective amount of a pharmaceutically acceptable gallium compound if the measured uptake of gallium radioisotope by the cancer tissue is greater than that of nearby healthy tissue.

2. The method of claim 1, wherein the gallium radioisotope is ⁶⁷Ga.

3. The method of claim 2, wherein the ⁶⁷Ga is administered in the amount of approximately 1 to 5 MBq per Kg of body weight.

4. The method of claim 1, wherein the measured uptake of gallium radioisotope by the cancer tissue is at least approximately ten percent higher than that of nearby healthy tissue.

5. The method of claim 4, wherein the measured uptake of gallium radioisotope by the cancer tissue is at least approximately twice that of nearby healthy tissue.

6. The method of claim 5, wherein the measured uptake of gallium radioisotope by the cancer tissue is at least approximately ten times that of nearby healthy tissue.

7. The method of claim 6, wherein the measured uptake of gallium radioisotope by the cancer tissue is at least approximately one hundred times that of nearby healthy tissue.

8. The method of claim 1, wherein the gallium scan is performed approximately 18 hours to 96 hours following administration of the gallium radioisotope.

9. The method of claim 8, wherein the gallium scan is performed approximately 24 hours to 72 hours following administration of the gallium radioisotope.

10. The method of claim 1, wherein the pharmaceutically acceptable gallium compound is gallium maltolate.

11. The method of claim 1, wherein the pharmaceutically acceptable gallium compound is gallium nitrate.

12. The method of claim 1, wherein the pharmaceutically acceptable gallium compound is gallium 8-quinolinolate.

13. A method of treating cancer in a patient in need thereof, comprising:
(1) administering to a patient ⁶⁷Ga-citrate in the amount of approximately 1 to 5 MBq per Kg of body weight;
(2) performing a gallium scan on the patient approximately 24 to 72 hours following administration of the ⁶⁷Ga-citrate;
(3) measuring the ratio of ⁶⁷Ga uptake in cancer tissue to that in nearby healthy tissue from the gallium scan;
(4) treating the patient with a therapeutically effective amount of a pharmaceutically acceptable gallium compound if the measured uptake of gallium radioisotope by the cancer tissue is greater than that of nearby healthy tissue.

14. The method of claim 13, wherein the measured uptake of ⁶⁷Ga by the cancer tissue is at least approximately ten percent higher than that of nearby healthy tissue.

15. The method of claim 14, wherein the measured uptake of ⁶⁷Ga by the cancer tissue is at least approximately twice that of nearby healthy tissue.

16. The method of claim 15, wherein the measured uptake of ⁶⁷Ga by the cancer tissue is at least approximately ten times that of nearby healthy tissue.

17. The method of claim 16, wherein the measured uptake of ⁶⁷Ga by the cancer tissue is at least approximately one hundred times that of nearby healthy tissue.

18. The method of claim 13, wherein the pharmaceutically acceptable gallium compound is gallium maltolate.

19. The method of claim 13, wherein the pharmaceutically acceptable gallium compound is gallium nitrate.

20. The method of claim 13, wherein the pharmaceutically acceptable gallium compound is gallium 8-quinolinolate.
